(19) United States
(12) Patent Application Publication
(10) Pub. No.: US 2025/0003918 A1
(43) Pub. Date: Jan. 2, 2025

(54) ELECTROCHEMICAL APTAMER SENSORS WITH NON-MONOLAYER BLOCKING LAYERS

(71) Applicant: University Of Cincinnati, Cincinnati, OH (US)

(72) Inventor: Jason Charles Heikenfeld, Cincinnati, OH (US)

(21) Appl. No.: 18/712,513

(22) PCT Filed: Sep. 23, 2022
(86) PCT No.: PCT/US2022/044509

§ 371 Date: May 22, 2024

Related U.S. Application Data
(60) Provisional application No. 63/282,440, filed on Nov. 23, 2021.

(51) Int. Cl. G01N 27/327

(52) U.S. Cl. CPC G01N 27/3271

(57) ABSTRACT

A device and method for detecting the presence of, or measuring the concentration or amount of, at least one analyte in a sample fluid, including (1) at least one electrode, (2) a plurality of affinity-based probes, at least one of the affinity-based probes being capable of binding to an analyte, (3) a plurality of redox molecules, wherein one or more affinity-based probes of the plurality of affinity-based probes each have at least one redox molecule associated therewith; and (4) a non-monolayer blocking layer associated with a surface of the at least one electrode.

### CROSS-REFERENCE TO RELATED APPLICATIONS

This application claims priority to, and the benefit of the filing date of, U.S. Application Ser. No. 63/282,440, titled “Electrochemical Aptamer Sensors with Non-monolayer Blocking Layers,” which was filed on Nov. 23, 2021—the disclosure of which is incorporated by reference herein in its entirety.

### FIELD OF THE INVENTION

This invention relates generally to aptamer sensors.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Electrochemical aptamer sensors can identify the presence and/or concentration of an analyte of interest via the use of an aptamer sequence that specifically binds to the analyte of interest. These sensors include aptamers attached to an electrode, wherein each of the aptamers has a redox active molecule (redox tag) attached thereto. The redox tag can transfer electrical charge to or from the electrode. When an analyte binds to the aptamer, the aptamer changes shape, bringing the redox tag closer to or further from the electrode. Over a plurality of aptamers (each having a redox tag) in the presence of a plurality of molecules of the analyte of interest, the redox tags will be brought closer to (or further from), on average, the electrode. This results in a measurable change in electrical current that can be translated to a measure of presence or concentration of the analyte. When used in this manner, then, aptamers are an example of an affinity-based biosensor.

A major unresolved challenge for aptamer sensors and other affinity-based biosensors (particularly those where the aptamers are bonded to the working electrode) is the lifetime of the sensors, especially for applications where continuous operation is required (“continuous” referring to multiple measurements over time by the same device). Such aptamer sensors are susceptible to degradation due to, among other things, desorption of the aptamers themselves from the electrode, and/or desorption of blocking molecules (such as mercaptohexanol) from the electrode. The aptamers and the blocking molecules together form a monolayer which can be referred to as a sensing monolayer. The blocking layer portion of the sensing monolayer is critical for (1) ensuring the aptamer can move freely and properly when changing conformation upon binding of analyte thereto, and (2) reducing electrical background current (including oxygen reduction current) and/or current due to electrochemical interference, which would otherwise wash-out the measured signal from the interaction of redox tag and electrode. The blocking layer, then, is important for achieving an accurate measurable response to analyte presence and/or concentration.

Current methods of fabrication of these aptamer sensor devices use a very simple and convenient approach of forming a partial sensing monolayer by thiol bonding aptamer to a gold electrode [via incubation of the electrode in solution including aptamer(s)], followed by forming a more complete sensing monolayer including the blocking molecule such as mercaptohexanol (via incubation of the electrode in mercaptohexanol solution). The use of mercaptohexanol in current sensors has been beneficial because, not only does a monolayer of mercaptohexanol reduce background current, but mercaptohexanol monolayers as-typically-formed have defects which allow for electron transfer between the redox tag and the electrode, these defects being few and/or small enough to still allow for minimization of oxygen reduction current and other major sources of background current.

Thus, researchers have had at their disposal a very ‘convenient’ way to make aptamer sensors for research applications. However, most researchers have not been motivated to address the longevity (or lack thereof) of aptamer sensors, and the same monolayer approach that is so convenient is also inherently fragile because the monolayer is able to desorb over time. Part of the cause for desorption is that each portion of the monolayer is a single molecule that has a single bond to the electrode, and statistically or energetically breaking one of these bonds with the electrode is not that difficult, especially at elevated temperatures such as body temperature. Although alternate approaches using different chemicals for the self-assembled monolayer have been tested or considered, the focus by researchers has continued to be on improving the chemistry of self-assembled monolayers themselves versus much more novel approaches to the problem of fragility and longevity for aptamers sensors. Novel approaches for electrochemical aptamer sensors which eliminate the drawbacks described above by not entirely relying on current monolayer technology could provide significant increases in longevity of aptamer sensors (and other affinity-based biosensors using such blocking layers).

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with biofluid and analytes.

One aspect of the present invention is directed to a device for detecting the presence of, or measuring the concentration or amount of, at least one analyte in a sample fluid. In this aspect, the device includes (1) at least one electrode, (2) a plurality of affinity-based probes, at least one of the affinity-based probes being capable of binding to an analyte, (3) a plurality of redox molecules, wherein one or more affinity-based probes of the plurality of affinity-based probes each have at least one redox molecule associated therewith; and (4) a non-monolayer blocking layer associated with a surface of the at least one electrode. In the device, the detection or measurement of any analyte may be caused by analyte binding to the affinity-based probe, which further causes a change in electron transfer from at least one redox molecule of the plurality of redox molecules. More particularly, the conformation of the affinity-based probe (e.g., an aptamer) changes upon binding to analyte in a manner the brings the redox molecule(s) closer to, or further from, the electrode.

Another aspect of the present invention is directed to a method of preparing a device for detecting the presence of, or measuring the concentration or amount of, at least one analyte in a sample fluid. In one embodiment the method includes coating an electrode with a layer that includes at least a plurality of affinity-based probes and a non-monolayer blocking layer. In certain embodiments, this may comprise (1) attaching a plurality of affinity-based probes to an electrode, and (2) forming a non-monolayer blocking layer on at least a portion of a surface of the electrode. Other embodiments of this aspect comprise (1) forming a non-monolayer blocking layer on at least a portion of a surface of an electrode, and (2) attaching a plurality of affinity-based probes to the non-monolayer blocking layer. Another embodiment of this aspect includes forming an anti-fouling layer onto the non-monolayer blocking layer.

### BRIEF DESCRIPTION OF THE DRAWINGS

The objects and advantages of the disclosed invention will be further appreciated in light of the following detailed descriptions and drawings in which:

FIG. 1A is a schematic of one embodiment of a conventional prior art sensor device.

FIG. 1B is a schematic of another embodiment of a conventional prior art sensor device.

FIG. 2 is a schematic of one embodiment of a device in accordance with principles of the present invention.

FIG. 3 is a schematic of another embodiment of a device in accordance with principles of the present invention.

FIG. 4 is a schematic of yet another embodiment of a device in accordance with principles of the present invention.

FIG. 5 is a graph showing the results of a test for measuring the stability of a mercaptohexanol blocking layer (monolayer) over time.

FIG. 6 is a graph showing the results of a test for measuring the stability of a SiO₂ blocking layer (non-monolayer) over time.

### DEFINITIONS

As used herein, “continuous sensing” with a “continuous sensor” means a sensor that changes in response to changing concentration of at least one solute in a solution such as an analyte. Similarly, as used herein, “continuous monitoring” means the capability of a device to provide multiple measurements of an analyte over time.

As used herein, the term “about,” when referring to a value or to an amount of mass, weight, time, volume, pH, size, concentration or percentage is meant to encompass variations of ±20% in some embodiments, ±10% in some embodiments, ±5% in some embodiments, ±1% in some embodiments, ±0.5% in some embodiments, and ±0.1% in some embodiments from the specified amount, as such variations are appropriate to perform the disclosed method.

As used herein, the term “electrode” means any material that is electrically conductive such as gold, platinum, nickel, silicon, conductive liquid infused materials such as ionic liquids, PEDOT:PSS, conductive oxides, carbon, boron-doped diamond, nanotubes or nanowire meshes, or other suitable electrically conducting materials.

As used herein, the term “blocking layer” means a homogeneous or heterogeneous layer of material or of one or more types of molecules on an electrode which reduce electrochemical background current and/or current due to electrochemical interference, and which may promote proper freedom of movement for the aptamer which is required for creating a measurable response to analyte concentration.

As used herein, the term “non-monolayer blocking layer” means a homogeneous or heterogeneous layer of material or of one or more types of molecules on an electrode which do not represent a monolayer configuration, and which reduces electrochemical background current and/or current due to electrochemical interference, and which may promote proper freedom of movement for the aptamer which is required for creating a measurable response to analyte concentration. For example, a metal or semiconductor oxide can be a non-monolayer blocking layer, or a thin polymer film may be a non-monolayer blocking layer, because they are comprised of multiple layers of atoms or molecules. A single atomic monolayer of SiO₂ for example would be a monolayer, whereas 3 nm of SiO₂ is a non-monolayer.

As used herein, the term “antifouling layer” means a homogeneous or heterogeneous layer of material or of one or more types of molecules on a surface which reduces fouling on a surface compared to if such an antifouling layer was not utilized.

As used herein, the term “endogenous antifouling layer” means a homogeneous or heterogeneous layer of endogeneous material or of one or more types of endogeneous molecules found in a sample that foul onto a surface such that further fouling is reduced or mitigated. Endogenous molecules, for example, could be contaminants in river water that is being measured, or for example proteins and peptides in interstitial fluid.

As used herein, the term “aptamer” means a molecule that undergoes a conformation or binding change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers and other affinity-based probes. Modifications may include substituting unnatural nucleic acid bases for natural bases within the aptamer sequence, replacing natural sequences with unnatural sequences, or other suitable modifications that improve sensor function, but which behave analogous to traditional aptamers. Two or more aptamers bound together can also be referred to as an aptamer (i.e., not separated in solution). Aptamers can have molecular weights of at least 1 kDa, 10 kDa, or 100 kDa.

As used herein, the term “redox tag” or “redox molecule” means any species such as small or large molecules with a redox active portion that when brought adjacent to an electrode can reversibly transfer at least one electron with the electrode. Redox tag or molecule examples include methylene blue, ferrocene, quinones, or other suitable species that satisfy the definition of a redox tag or molecule. In some cases, a redox tag or molecule is referred to as a redox mediator. Redox tags or molecules may also exchange electrons or change in behavior when brought into proximity with other redox tags or molecules. Exogenous redox molecules are those added to a device, e.g., they are not endogeneous and provided by the sample fluid to be tested.

As used herein, the term “change in electron transfer” means a redox molecule whose electron transfer with an electrode has changed in a measurable manner. This change in electron transfer can, for example, originate from availability for electron transfer, distance from an electrode, diffusion rate to or from an electrode, a shift or increase or decrease in electrochemical activity of the redox molecule, or any other embodiment as taught herein that results in a measurable change in electron transfer between the redox molecule and the electrode.

As used herein, the term “sensing monolayer” means at least a plurality of aptamers on a working electrode, which may also include a plurality of molecules or mixtures of molecules that form a blocking layer or an anti-fouling layer.

As used herein, the term “analyte” means any solute in a solution or fluid which can be measured using a sensor. Analytes can be small molecules, proteins, peptides, electrolytes, acids, bases, antibodies, molecules with small molecules bound to them, DNA, RNA, drugs, chemicals, pollutants, or other solutes in a solution or fluid.

As used herein, the term “continuous sensing” simply means the device records a plurality of readings over time. Even a point-of-care testing device which provides a single data point can be considered a continuous sensing device if, for example, it is a 15 minute test, that operates by taking multiple data points over 15 minutes and averaging them to provide a single data measure.

As used herein, a “device” comprises at least one sensor based on at least one aptamer, at least one sensor solution, and at least one sample solution. Devices can sense multiple samples and be in multiple configurations such as a device to measure a pin-prick of blood, or a microneedle or in-dwelling sensor needle to measure interstitial fluid, or a device to measure saliva, tears, sweat, or urine sensor, or a device to measure water pollutants or food processing solutes, or other devices which measure at least one analyte found in a sample solution.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Certain embodiments of the disclosed invention show sensors as simple individual elements. It is understood that many sensors require two or more electrodes, reference electrodes, or additional supporting technology or features which are not captured in the description herein. Sensors can be in duplicate, triplicate, or more, to provide improved data and readings. Sensors may provide continuous or discrete data and/or readings. Certain embodiments of the disclosed invention show sub-components of what would be sensing devices with more sub-components needed for use of the device in various applications, which are known (e.g., a reference or counter electrode, a battery, antenna, adhesive), and for purposes of brevity and focus on inventive aspects, such components may not be explicitly shown in the diagrams or described in the embodiments of the disclosed invention. All ranges of parameters disclosed herein include the endpoints of the ranges.

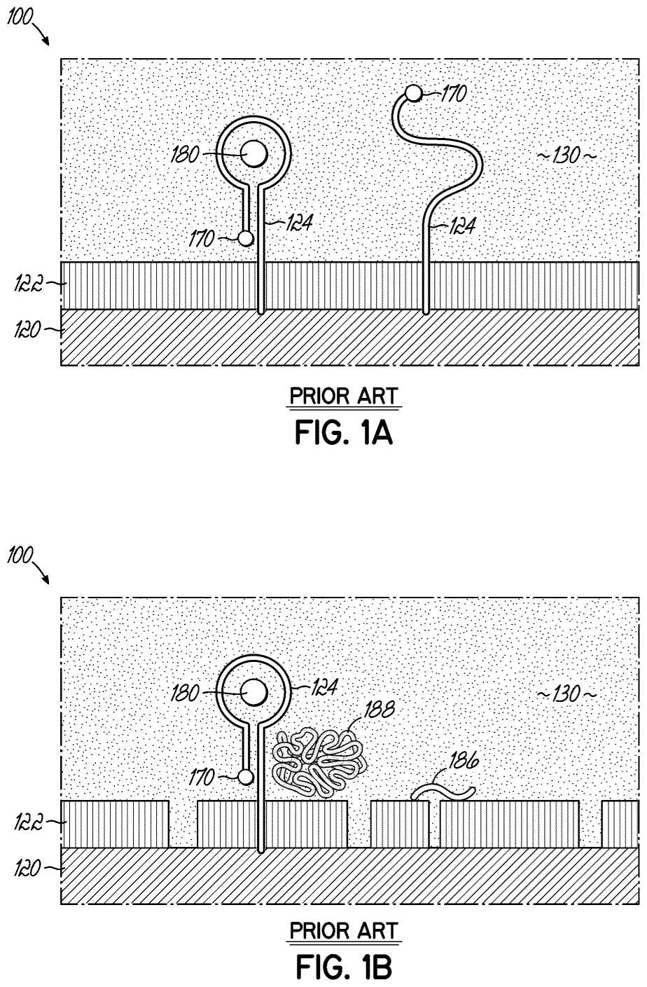

With reference to FIGS. 1A and 1B, a conventional prior art sensor device 100 as placed initially in a sample fluid 130, such as interstitial fluid, is shown. The device 100 includes at least one working electrode 120 such as gold, carbon, or other suitable electrode material; at least one monolayer blocking layer 122 [the blocking layer may include (1) a plurality of molecules such as mercaptohexanol or hexanethiol that are thiol bonded to the electrode, or (2) a plurality of natural solutes in blood that can act as a blocking layer, or (3) other suitable molecules depending on application and on the choice of electrode 120 material]; at least one aptamer 124 that is responsive to binding to an analyte 180; and a redox tag 170, such as methylene blue, associated with the at least one aptamer, such as by being bound thereto. In the generic example taught for FIGS. 1A and 1B, the aptamer 124 is a simple stem loop (hairpin) aptamer where analyte 180 binding causes the stem loop to form and the redox current measured from the redox tag 170 to increase, as measured using square wave voltammetry, chronoamperometry, or other suitable technique. In absence of analyte 180 binding to the aptamer 124 the stem loop conformation does not form and the redox current thus does not increase. Thus, changes in a measurement of electrical redox current can be used as a signal to interpret changes in concentration of the analyte 180.

With further reference to FIG. 1B, a challenge with aptamer sensors is that when placed into initial operation the sample fluid 130, over a period of tens of minutes to hours, the signal (e.g., redox current) initially decreases by 30%, 50%, or even more, due to effects such as fouling by small molecules 186, proteins 188, or other solutes in the sample fluid 130, but also due to desorption of the sensing monolayer, including aptamer 124 and/or blocking layer 122.

As can be seen, current sensor devices include several drawbacks and limitations, particularly resulting from the blocking layers found on such current devices. Various aspects of the present invention, however, resolve such drawbacks and limitations (including the amount of initial or longer term desorption, instability, and/or fouling of the sensing monolayer). In that regard, one aspect of the present invention is directed to a device for detecting the presence of, or measuring the concentration or amount of, at least one analyte in a sample fluid. In certain embodiments of this aspect, the device includes (1) at least one electrode, (2) a plurality of affinity-based probes, at least one of the affinity-based probes being capable of binding to an analyte, (3) a plurality of redox molecules, wherein one or more affinity-based probes of the plurality of affinity-based probes each have at least one redox molecule associated therewith; and (4) a non-monolayer blocking layer associated with a surface of the at least one electrode. In the device, the detection or measurement of any analyte may be caused by analyte binding to the affinity-based probe, which further causes a change in electron transfer from at least one redox molecule of the plurality of redox molecules. For example, the conformation of the affinity-based probe (e.g. an aptamer) changes upon binding to analyte in a manner that brings the redox molecule(s) closer to, or further from, the electrode.

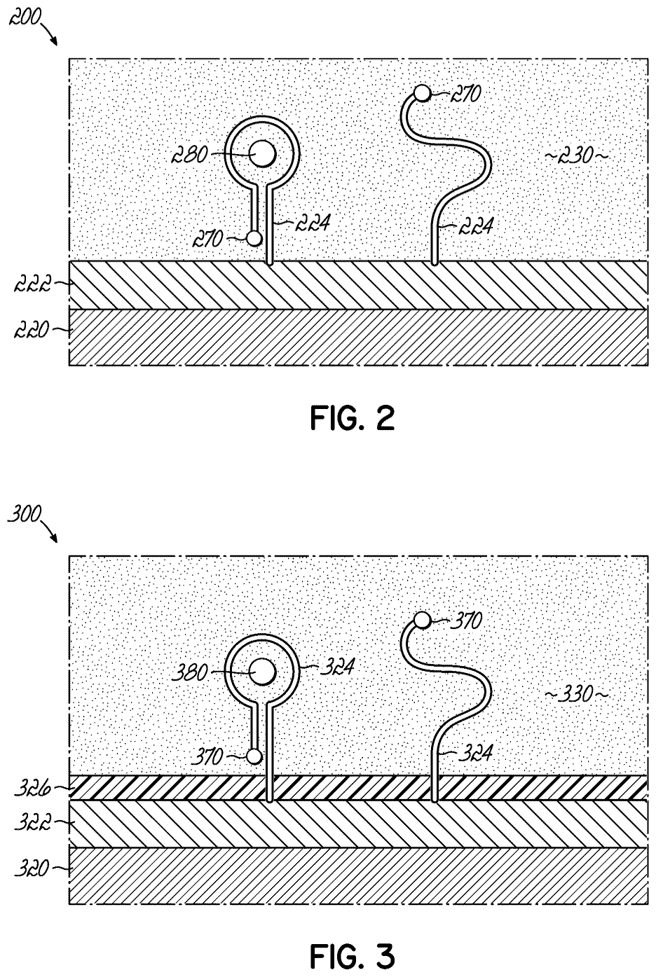

With reference now to FIG. 2, where like numerals refer to like features, an example of a device in accordance with principles of the invention is shown. In this embodiment the device 200 includes a non-monolayer blocking layer 222 to which an aptamer 224 is attached. The non-monolayer blocking layer 222 is positioned adjacent to at least one electrode 220. The non-monolayer blocking layer 222 may be formed from one or more materials including, but not limited to, a metal oxide, a semiconductor oxide, a thin polymer film, acrylic, polyamide, an inorganic dielectric, a hydrogel, a fluoropolymer, parylene C, parylene HT, PVDF, silicon dioxide, silicon nitride, titanium dioxide, and barium titanate. For example, the non-monolayer blocking layer 222 could be, for example acrylic, polyamide, an inorganic dielectric such as SiON, or other suitable blocking material. The example non-monolayer blocking layer 222 shown in FIG. 2 may itself be resistant to fouling, and thus not require a separate antifouling layer, and/or the non-monolayer blocking layer 222 may be allowed to foul (not shown), thereby forming an endogenous anti-fouling layer.

With reference to FIG. 3, where like numerals refer to like features, a device 300 includes a non-monolayer blocking layer 322 to which an aptamer 324 is attached. The non-monolayer blocking layer 322 is positioned adjacent to at least one electrode 320. The non-monolayer blocking layer 222 may be formed from one or more materials, such as those described above with respect to the embodiment of FIG. 2. The device 300 also includes an antifouling layer 326 that is positioned adjacent to the non-monolayer blocking layer 322, in the illustrated embodiment. The antifouling layer 326 may be, for example, a solid layer of material or a monolayer terminated with polyethylene glycol attached to non-monolayer blocking layer 322. In one embodiment, the antifouling layer 326 may be formed from a zwitterionic material that is bound to layer 322. In one embodiment, the antifouling layer 326 may be formed from exogenous material (i.e., the material is from a source external to the device in the test environment—e.g. the material is not endogeneous and provided from within the sample fluid to be tested). In other embodiments, described in greater detail below, the material of the antifouling layer may be endogenous.

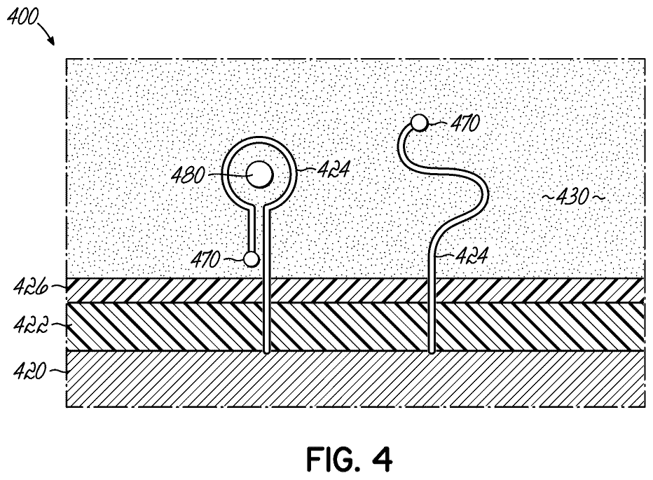

With reference to FIG. 4, where like numerals refer to like features, the device 400 includes electrode 420, non-monolayer blocking layer 422 and aptamer(s) 424. In this embodiment, the aptamer 424 is attached to electrode 420 (whereas in embodiments shown in FIGS. 2 and 3, the aptamer was attached to the non-monolayer blocking layer). In the embodiment of FIG. 4, the aptamer 424 may be attached to electrode 420 followed by addition of non-monolayer blocking layer 422 and antifouling layer 426, for example by electrodeposition of a polymer or oxidation or anodization of an electrode. Alternatively, aptamer 424 could be attached after non-monolayer blocking layer 422 and antifouling layer 426 have started to be added (not shown) and therefore partially embedded in one of materials of non-monolayer blocking layer 422 or antifouling layer 426, but not attached directly to electrode 420.

The example embodiments described above for FIGS. 2, 3, and 4 can also be extended to other forms of affinity-based electrochemical sensors that benefit from blocking layers that are not specifically illustrated or described herein, including protein-catalyzed capture agents, peptide-based biosensors, immunosensors, and others. In some cases, the amount of redox active species used for detection can also be much higher in concentration (10×, 100×, or more) than the redox species used for aptamers such that specific parameters taught herein can be further extended, for example in redox signal strength from a redox marker compared to background current due to oxygen reduction.

In the example embodiments described above for FIGS. 2, 3, and 4, the non-monolayer blocking layer 222, 322, 422 is superior in performance, longevity, antifouling, or at least one other performance factor compared to a monolayer blocking layer such as mercaptohexanol, hexanethiol, peptides with cysteine groups for thiol bonding to the gold, and other monolayer blocking layers. For example, a non-monolayer blocking layer 222, 322, 422 can be longer-lasting because it is formed of molecules that are not only attached to an adjacent surface (such as surface of the electrode 220, 320, 420), but the molecules are also attached to each other. As a result, the non-monolayer blocking layer 222, 322, 422 is unlikely to desorb partially or completely. Further, a non-monolayer blocking layer 222, 322, 422 can have superior electrochemical behavior by, for example, blocking heterogeneous sources of charge transfer (oxygen reduction, interferents in the fluid 230, 330, 430 etc.) while promoting strong charge transfer between the redox tag 270, 370, 470 and electrode 220, 320, 420. And, a non-monolayer blocking layer 222, 322, 422 can have superior anti-fouling resistance by, for example, having an hydrophilic and/or charged surface adjacent to fluid 230, 330, 430. Or, as another example (particular to the embodiments of FIGS. 3 and 4), non-monolayer blocking layer 322, 422 can have superior antifouling resistance by having a layer 326, 426 (shown in FIGS. 3 and 4) adjacent to fluid 330, 430 that has superior anti-fouling properties compared to a monolayer blocking layer such as hydrophobic hexane thiol.

The example embodiments described above for FIGS. 2, 3, and 4 will now be taught in greater detail in terms of electrical and thickness characteristics for the non-monolayer blocking layer. These characteristics are non-limiting and by example only to assist in teaching embodiments of the invention. A first characteristic is electrical capacitance. A non-monolayer blocking layer should generally not have a capacitance that is so large that techniques such as square-wave voltammetry cannot be utilized. For example, an alky thiol monolayer blocking layer with a dielectric constant of ˜2.6 and ˜0.7 nm or 1.4 nm thickness can have an electrical capacitance C=e*eo*A/t of C=2.6*8.854E-14 F/cm/7E-8 cm=3.3 μF/cm² or C=e*eo*A/t of C=2.6*8.854E-14 F/cm/1.4E-7 cm=1.6 μF/cm² The electrical double layer capacitance in most fluids (such as fluids 230, 330, 430) is so large that it is negligible in this calculation (due to thinness of the double layer in high salt conditions such as biofluids and the very high dielectric constant of water). This capacitance allows for square-wave voltammetry to be reliably performed generally up to ˜1 kHz. Therefore, a non-monolayer blocking layer, such as layer 222, 322, 422, may have a capacitance that is at least one of less than 200 μF/cm², less than 20 μF/cm², less than 10 μF/cm², less than 5 μF/cm², or less than 3 μF/cm². For example, 200 μF/cm² could limit robust square wave voltammetry measurement frequency to −10 Hz maximum and/or amperometrically or chronoamperometrically measured signals to those with slow electron transfer rates and/or weak signals. Materials and dielectric constants (˜e) may include, for example a fluoropolymer (˜2), polymer such as acrylic (˜3) or parylene C (˜3) or Parylene HT (˜2) or PVDF (˜8), silicon dioxide (˜4), silicon nitride (˜10), or titanium dioxide (˜80), or barium titanate (˜1000).

Consider an example of a sensor having a non-monolayer blocking layer with a dielectric constant of 4. To maintain less than 20 μF/cm², the thickness of the non-monolayer blocking layer would need to be at least t=e*eo/C=4*8.853E-14 F/cm/20E-6 F/cm²=1.8E-8 cm, or at least less 0.18 nm thick. Such a thickness is very thin, given a general rule of ˜0.1 nm per molecular bond. Furthermore, tunneling current is proportional to applied voltage and exponentially proportional to decreasing thickness and can begin to become significant (breakdown current less so for such thin films and due to a requirement of being ˜6 times the electronic bandgap voltage of the layer). Also, a thinner non-monolayer blocking layer can also be so thin that electrical screening of the aptamer is increased and the aptamers net negative charge causes greater repulsion from the surface during electrical measurement. Therefore, more generally, reliability and background current (rather than capacitance) may inform a preferred thickness of the non-monolayer blocking layer to be at least several atoms/molecules thick and in different embodiments at least one of greater than 0.2 nm, greater than 0.5 nm, greater than 1 nm, or greater than 2 nm thick.

The example embodiments described above for FIGS. 2, 3, and 4 will now be taught in greater detail in terms of characteristics for defects (e.g., pores) associated with a non-monolayer blocking layer. A perfectly defect-free non-monolayer blocking layer with little or no pores and little or no tunneling current would generally make for a poor biosensor because electron transfer between the electrode 220, 230, 240 and the redox tag 270, 380, 480 would be blocked, and/or electron transfer kinetics too slow, which limits available measurement techniques and signal quality. A too highly defective non-monolayer blocking layer could have too much background current from molecular interferents in fluid 230, 330, 430 and/or due to oxygen reduction current. Therefore, a balance or ‘goldilocks’ zone for the right amount of defects is useful. This balance can be informed by use of existing alkyl thiol monolayers used in biosensors. For example, with decanethiolate monolayers, defect sizes are generally less than 0.3 nm in size on unannealed gold and generally less than 0.5 nm in size on annealed gold, occupying fractional area of the surface area generally less than 0.1, less than 0.05, or even less than 0.02. In different embodiments, non-monolayer blocking layer 222, 322, 422 has pores that are at least one of less than 1 nm, less than 0.5 nm, or less than 0.25 nm in size. Pores can allow for incorporation of ion/electron conductive species such as a water molecule (˜0.27 nm diameter) or small conjugated molecules. In different embodiments, non-monolayer blocking layer 222, 322, 422 has pores that have a fractional area of the total surface area that is less than 0.2, less than 0.1, less than 0.05, or even less than 0.02. In different embodiments, non-monolayer blocking layer 222, 322, 422 has pores that have a fractional area of the total surface area that is at least greater than 0.001, greater than 0.002, greater than 0.005, greater than 0.01, or greater than 0.05.

Defects can be created in numerous ways. For example, semi-insulating non-monolayer blocking layer defects can be at the location of semi-insulator dopants that create electronic conduction (such as III-V nitride semiconductors). Crystal grain (domain) boundaries in two or three dimensions can create defects or electrical conductive regions, similar to how two-dimensional defects are created in self-assembled monolayers. Non-monolayer blocking layers can be created with pores via, for example, the use of polymers that are deposited with a solvent that must escape during curing, or by creating pores during plasma-assisted deposition of a fluorocarbon layer or by depositing an inorganic dielectric at high gas pressures or rates which increases defectivity. Pores can be created using templating with molecules that are deposited before or co-deposited with non-monolayer blocking layer similar to how molecular imprinted polymers are fabricated (template molecules can be dissolved away such as salts, or etched away such as metals, or dissolved away or burned away such as organic molecules). An organic material such as Parylene HT or Parylene C can be deposited with a wide range of defects due to partial gas-phase reaction followed by deposition (controlled by deposition rate or vacuum pressure during deposition), or due to the inherent porosity of the polymer itself. Organic layers with porosity can be improved by capping with a less porous physical and/or chemical vapor or solution deposition SiO₂, Si₃N₄, Al₂O₃, AlN, or mixtures thereof (or the reverse, inorganic layer first, organic layer second). Some inorganic materials have inherent porosity such as spontaneously oxidized or anodized Al₂O₃. Non-monolayer blocking layers, such as those formed from metal oxides, may also have non-zero zeta potentials in the sample fluid. Zeta potential is a surface charge in solution that can enhance or dimmish switching of a device (for example a large negative zeta potential could repel a negatively charged aptamer and reduce signal strength). Zeta potential can therefore be optimized for each sensor and application, and for example be adjusted by having multi-layer non-monlayer blocking layers where the top layer facing sample has an optimal zeta potential and the lower layers have optimal electron-transfer and interferent blocking characteristics.

With further reference to embodiments of the present invention, a non-monolayer blocking layer can be formed from the underlying electrode material itself, for example by oxidizing silicon, aluminum, titanium, or other suitable materials. Sulfur, nitrogen, phosphorus, and other reactive species can also be used in place of oxygen to react an electrode to create wide-band-gap semiconductors or insulators. Hence, forming the non-monolayer blocking layer occurs via reaction with electrode.

The example embodiments described above for FIGS. 3 and 4 will now be taught in greater detail in terms of antifouling and passivation characteristics for a non-monolayer blocking layer 322, 422 provided by layer or material 326, 426. In one example, non-monolayer blocking layer 322, 422 can be a material with a negatively charged or hydrophilic end group that repels foulants and/or protects pores or defects in non-monolayer blocking layer 322, 422 from be occluded by solutes in fluid 330, 430. In another example, layer 326, 426 could be a self-assembled monolayer of amphiphilic molecules, which normally have the molecular structure of R₁—(CH₂)ₙ—R₂, where R₁ is a headgroup that prevents fouling, (CH₂)ₙ is in many examples a nonpolar alkane chain, and R₂ is an anchoring group that is chemically attached to non-monolayer blocking layer 322, 422. For different substrates, the selection of the anchoring group, R₂, will be different: thiol (—SH) may be used on gold or silver; silane (—SiCl₃) may be used on glass and silicone; and silane or phosphate may be used on metal oxides. These same attachment techniques may also be used to attach the aptamer 324, 424 to non-monolayer blocking layer 322, 422. Layer 326, 426 can also include a hydrogel, and can be thicker such that it encompasses the aptamer 324, 424 as long as the hydrogel is adequately porous to allow freedom of movement for the aptamer. Example hydrogels include biocompatible poly(hydroxyethyl methacrylate) and poly(ethylene glycol).

The example embodiments briefly described above for FIGS. 2, 3, and 4 will now be taught in greater detail in terms of characteristics of electrode 220, 320, 420. Electrode may be a material such as gold, platinum, aluminum, carbon, conducting polymer, boron-doped diamond or other suitable material. Diamond can be useful as it inhibits solvent interactions and oxygen reduction both which cause increased background current.

The example embodiments briefly described above for FIGS. 2, 3, and 4 will now be taught in greater detail in terms of general, preferred, and ideal electrochemical performance. A generally testable method to measure electrochemical performance would be to benchmark square wave voltammetry background current at a current of ˜0.1 to 10 μA/cm² and a redox peak current that is least 2, 5, or 10× greater than this background current. The above taught examples may achieve this performance.

Another aspect of the present invention is directed to a method of preparing a device for detecting the presence of, or measuring the concentration or amount of, at least one analyte in a sample fluid. In one embodiment the method includes coating an electrode with a layer that includes at least a plurality of affinity-based probes and a non-monolayer blocking layer. In certain embodiments, this may comprise (1) attaching a plurality of affinity-based probes to an electrode, and (2) forming a non-monolayer blocking layer on at least a portion of a surface of the electrode. Other embodiments of this aspect comprise (1) forming a non-monolayer blocking layer on at least a portion of a surface of an electrode, and (2) attaching a plurality of affinity-based probes to the non-monolayer blocking layer.

And so, the example embodiments described above for FIG. 4 will now be taught in greater detail in terms of fabrication techniques. Non-monolayer blocking layer 422 can be formed, at least in part, after aptamer 424 is attached to the device 400. Electrodeposition, such as used for molecular imprinted polymers to form an insulating layer around a template molecule, can be used as such a method to form device 400. Based on electrode choice (e.g., carbon, gold, etc.) non-monolayer blocking layer 422 could also include an endogenous layer of solutes found in fluid 430 such as amino acids, steroid hormones, peptides, and proteins. Alternately, non-monolayer blocking layer 422 could be deposited very thin (0.1's nm) and completed in thickness by a layer of endogenous solutes that initially foul the surface such that further fouling is prohibited or significantly reduced after the initial fouling has occurred (for example, after 2 hours of exposure to the endogenous solutes).

With further reference to embodiments of the present invention, although not specifically illustrated in FIGS. 2-4 the present invention may also apply to aptamers or other affinity-based probes in solution (e.g., aptamer, protein, or other probe that includes a change in availability of electron transfer from the redox tag when it binds to an analyte of interest). For example, an aptamer that folds upon itself with analyte binding and brings the redox tag more internal or ‘hidden’ inside the aptamer will generally have reduced electron transfer with an electrode even if the aptamer is freely in solution.

With further reference to embodiments of the present invention, although not specifically illustrated in FIGS. 2-4, in some cases where longevity is to be increased, fouling on the non-monolayer blocking layer may be self-cleaned or cleaned by another apparatus or material inside the device. For example, the non-monolayer blocking layer 222, 322, 422 could be formed of a wide bandgap semiconductor including alloys such as AlGaN or InAlGaN alloys, such that at measuring voltages the non-monolayer blocking layer is insulating but at higher voltages exceeding the breakdown field of the non-monolayer blocking layer (avalanche or tunneling breakdown), increased current flow can be used to electrochemically clean the surface of the non-monolayer blocking layer, and to fully or partially remove foulants from time to time as needed. Hot electrons and higher current can be controlled with thickness, voltage and crystallinity in such devices to tune the amount of energy available for self-cleaning at the layer surface, with example hot electron distributions as taught by Heikenfeld in ‘Multiple color capability from rare earth-doped gallium nitride’ Materials Science and Engineering B81 (2001) 97-101 (incorporated by reference herein). In yet another example, ceramic or other types of micro or nanobeads can be placed inside a device to mechanically abrade and remove fouling as the device is mechanically vibrated during use.

With further reference to embodiments of the present invention, although not specifically illustrated in FIGS. 2-4, in some cases non-monolayer blocking layer 222, 322, 422 is ideally deposited using a reaction-rate limited process (as opposed to a diffusion-rate limited deposition process such as physical vapor deposition). As an example, atomic layer deposition can be used to deposit a highly defect-free dielectric, and defects can be increased by decreasing the deposition temperature, which results in partial deposition and organic residues on the surface, thereby allowing tuning of defect density based on deposition temperature. A reaction rate limited process may also be needed for porous electrodes, to allow penetration and uniform coating inside the porous structure. Reaction rate limited deposition methods may also include layer-by-layer self-assembly methods in solution. Layer-by-layer deposition has also been demonstrated for polymers in vapor phase reactions as well, as taught in ‘Moon, H., Seong, H., Shin, W. et al. Synthesis of ultrathin polymer insulating layers by initiated chemical vapour deposition for low-power soft electronics. Nature Mater 14, 628-635 (2015). https://doi.org/10.1038/nmat4237′ (incorporated by reference herein).

### Example

A conventional mercaptohexanol (MCH) blocking layer (i.e., a monolayer blocking layer) was prepared on a gold electrode and compared with 1 nm of e-beam deposited SiO₂ as an inorganic non-monolayer blocking layer also on a gold electrode—to determine performance of each blocking layer in blocking background current over time (which correlates to longevity of a sensor having such blocking layers).

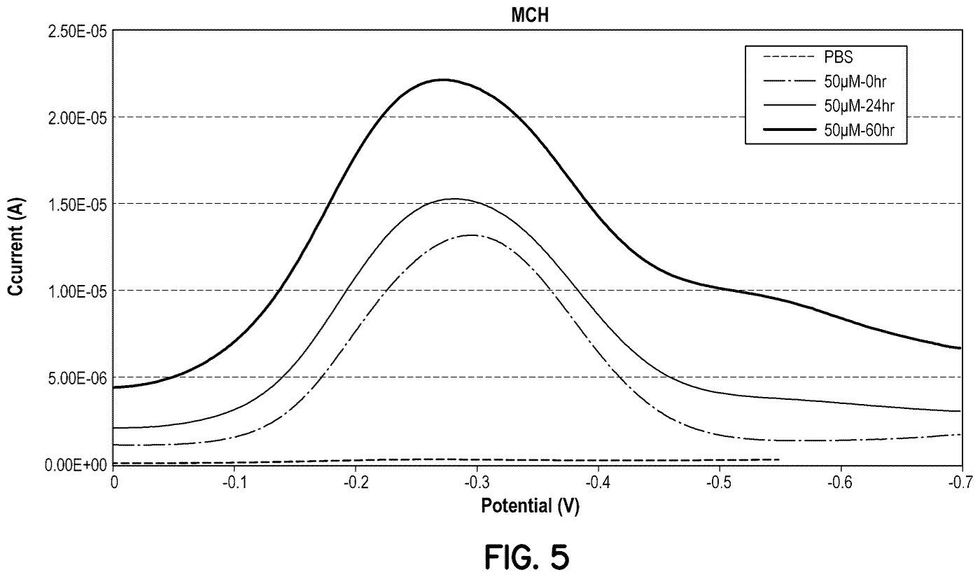

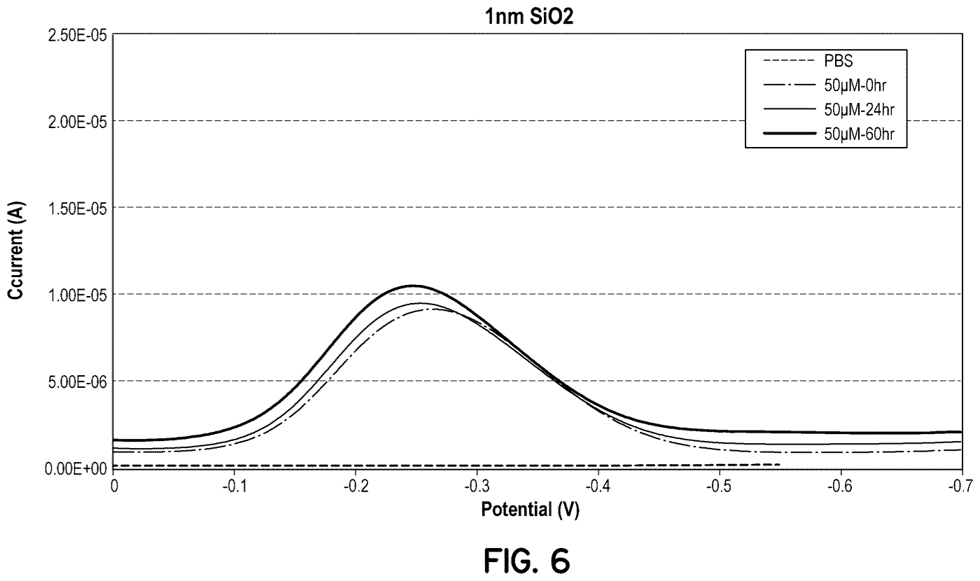

Tests were performed (1) in a buffer solution (PBS), and (2) with 50 μM of a solution-phase redox mediator of hexaammineruthenium trichloride added to the buffer solution. All tests were performed at a temperature of at least room temperature (20 degrees Celcius). Scans were measured using square wave voltammetry. Potential versus current was determined at 0 hr, 24 hr, and 60 hr. The results are shown in FIG. 5 (for MCH blocking layer) and FIG. 6 (for SiO₂ blocking layer). As can be seen, the non-monolayer blocking layer (SiO₂) exhibited superior performance in stability and longevity compared to the monolayer blocking layer (MCH). The SiO₂ blocking layer performs well even up to 60 hours of testing and increased background current due to oxygen reduction is minimal at most (as observable as a baseline current increase at the more negative potentials applied). The non-monolayer blocking layer clearly exhibits greater stability of operation than the MCH blocking layer, even after 60 hours of operation (results for the non-monolayer blocking layer at 60 hr are very similar to those at 24 hr and 0 hr—whereas the results for MCH monolayer worsen over passing time). This example is simply to show example operation and fundamental advantages of a non-monolayer blocking layer, and is not meant to illustrate full sensor device function as taught herein.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A device for detecting the presence of, or measuring the concentration or amount of, at least one analyte in a sample fluid, the device comprising:
at least one electrode;
a plurality of affinity-based probes, at least one of the affinity-based probes being capable of binding to an analyte;
a plurality of redox molecules, wherein one or more affinity-based probes of the plurality of affinity-based probes each have at least one redox molecule associated therewith; and
a non-monolayer blocking layer associated with a surface of the at least one electrode.

2. The device of claim 1, wherein each affinity-based probe of at least a subset of the plurality of affinity-based probes is an aptamer.

3. The device of claim 1, wherein the plurality of affinity-based probes are attached to the at least one electrode.

4. The device of claim 1, wherein the plurality of affinity-based probes are attached to the non-monolayer blocking layer.

5. The device of claim 1, wherein redox molecules of the plurality of redox molecules include a material chosen from methylene blue, ferrocene, quinones, and other suitable species.

6. The device of claim 1, wherein the electrode includes a material chosen from gold, platinum, nickel, aluminum, silicon, conductive liquid infused materials such as ionic liquids, PEDOT:PSS, conductive oxides, carbon, boron-doped diamond, nanotubes or nanowire meshes, and other suitable electrically conducting materials.

7. The device of claim 1, wherein the non-monolayer blocking layer includes a plurality of molecules that are attached both to the electrode and to at least one other molecule of the plurality of molecules.

8. The device of claim 1, wherein the non-monolayer blocking layer includes at least in part material selected from the group of a metal oxide, a semiconductor oxide, a thin polymer film, acrylic, polyamide, an inorganic dielectric, a hydrogel, a fluoropolymer, parylene C, parylene HT, PVDF, silicon dioxide, silicon nitride, titanium dioxide, and barium titanate.

9. The device of claim 1, wherein the non-monolayer blocking layer includes a material that is an inorganic dielectric, and wherein the inorganic dielectric of a metal oxide or a semiconductor oxide.

10. The device of claim 1, wherein the non-monolayer blocking layer includes a material that is a hydrogel, and wherein the hydrogel is chosen from poly(hydroxyethyl methacrylate) and poly(ethylene glycol).

11. The device of claim 1, wherein the non-monolayer blocking layer is a homogeneous layer.

12. The device of claim 1, wherein the non-monolayer blocking layer is a heterogeneous layer.

13. The device of claim 1, wherein the non-monolayer blocking layer includes a material that is resistant to fouling.

14. The device of claim 13, wherein the non-monolayer blocking layer includes a wide bandgap semiconductor.

15. The device of claim 14, wherein the wide bandgap semiconductor includes an alloy chosen from AlGaN, InAlGaN, or mixtures thereof.

16. The device of claim 1, further comprising at least one feature to remove surface fouling during use.

17. The device of claim 1, wherein the non-monolayer blocking layer includes a hydrophilic and/or charged surface.

18. The device of claim 17, wherein the hydrophilic and/or charged surface is positioned on the device to be adjacent to fluid when the device is exposed to fluid.

19. The device of claim 1, further comprising at least one anti-fouling layer.

20. The device of claim 19, wherein the antifouling layer is an endogenous antifouling layer.

21. The device of claim 19, wherein the antifouling layer is an exogenous antifouling layer.

22. The device of claim 21, further comprising at least in part polyethylene glycol for the exogenous antifouling layer.

23. The device of claim 19, wherein the antifouling layer comprises zwitterionic molecules.

24. The device of claim 21, wherein antifouling layer is comprised of a plurality of molecules that have a molecular structure of R₁—(CH₂)ₙ—R₂, wherein R₂ is chemically attached to blocking layer.

25. The device of claim 19 wherein the antifouling layer comprises charged molecules.

26. The device of claim 21, wherein the blocking layer includes a material chosen from metal or semiconductor oxides, wherein the exogenous antifouling layer is attached to the blocking layer using a silane (—SiCl₃) group.

27. The device of claim 24, wherein the blocking layer includes metal oxides, and R₂ is chosen from silane or phosphate.

28. The device of claim 1, wherein the non-monolayer blocking layer has an electrical capacitance measured at less than 1 kHz.

29. The device of claim 28, wherein the non-monolayer blocking layer has an electrical capacitance selected from the group consisting of less than 200 μF/cm², less than 20 μF/cm², less than 10 μF/cm², less than 5 μF/cm², and less than 3 μF/cm².

30. The device of claim 1, wherein the non-monolayer blocking layer has a thickness that is on average a value selected from the group consisting of at least >0.2, >0.5, >1, and >2 nm thick.

31. The device of claim 1, wherein the non-monolayer blocking layer has a plurality of pores or defects that promote electron transfer between the electrode and the redox molecule, and said pores on average are less than a value selected from the group consisting of at least <1, <0.5, and <0.25 nm in size.

32. The device of claim 1, wherein the non-monolayer blocking layer has a plurality of pores or defects that promote electron transfer between the electrode and the redox molecule, and said pores on average have a fractional area of the total surface area of the sensor that is selected from the group consisting of <0.2, <0.1, <0.05, and less than 0.02 but at least greater than a value selected from the group consisting of at least >0.001, >0.002, >0.005, >0.01, and >0.05.

33. The device of claim 1, wherein the non-monolayer blocking layer limits the electrical background current as measured by square wave voltammetry and has a redox peak current that is a value selected from the group consisting of at least 2, 5, and 10× greater than this background current.

34. The device of claim 1, wherein the non-monolayer blocking layer is comprised of a material with an electrical breakdown field, and during measurements the applied electric field is below said breakdown field, and the applied electric field can periodically be applied above the breakdown field to reduce or remove fouling of the surface of the non-monolayer blocking layer.

35. The device of claim 1, wherein the non-monolayer blocking layer is comprised of a material, said material being deposited using a reaction-rate limited method.

36. A method of preparing a device for detecting the presence of, or measuring the concentration or amount of, at least one analyte in a sample fluid, the method comprising:
coating an electrode with a layer that includes at least a plurality of affinity-based probes and a non-monolayer blocking layer.

37. The method of claim 36, wherein forming the non-monolayer blocking layer occurs via deposition of a material.

38. The method of claim 36, wherein forming the non-monolayer blocking layer occurs via reaction with electrode.

39. The method of claim 36, wherein coating of affinity-based probes occurs after coating of a non-monolayer blocking layer.

40. The method of claim 36, wherein coating of affinity-based probes occurs before coating of a non-monolayer blocking layer.

41. The method of claim 36, wherein forming the non-monolayer blocking layer occurs via deposition using a reaction-rate limited process.

42. The method of claim 36, wherein one or more aptamers of the plurality of aptamers each have at least one redox molecule associated therewith.

43. The method of claim 36, further comprising forming an antifouling layer adjacent to the non-monolayer blocking layer.

44. The method of claim 36, wherein forming a non-monolayer blocking layer on at least a portion of a surface of the electrode further comprises forming defects in the non-monolayer blocking layer.

45. The method of claim 44, wherein the defects are pores.

46. The method of claim 36, wherein the non-monolayer blocking layer is comprised at least in part of a metal oxide or semiconductor oxide.

47. A method of preparing a device for detecting the presence of, or measuring the concentration or amount of, at least one analyte in a sample fluid, the method comprising:
forming a non-monolayer blocking layer adjacent to an electrode; and
forming a plurality of affinity-based probes adjacent to an electrode.

48. The method of claim 47, wherein forming the non-monolayer blocking layer occurs via deposition of a material.

49. The method of claim 47, wherein forming the non-monolayer blocking layer occurs via reaction with electrode.

50. The method of claim 47, wherein coating of affinity-based probes occurs after coating of a non-monolayer blocking layer.

51. The method of claim 47, wherein coating of affinity-based probes occurs before coating of a non-monolayer blocking layer.

52. The method of claim 47, wherein forming the non-monolayer blocking layer occurs via deposition using a reaction-rate limited process.

53. The method of claim 47, wherein one or more aptamers of the plurality of aptamers each have at least one redox molecule associated therewith.

54. The method of claim 47, further comprising forming an antifouling layer adjacent to the non-monolayer blocking layer.

55. The method of claim 47, wherein forming a non-monolayer blocking layer on at least a portion of a surface of the electrode further comprises forming defects in the non-monolayer blocking layer.

56. The method of claim 47, wherein the defects are pores.

57. The method of claim 47, wherein the non-monolayer blocking layer is comprised at least in part of a metal oxide or semiconductor oxide.
